(19) United States
(12) Patent Application Publication
(10) Pub. No.: US 2025/0387588 A1
(43) Pub. Date: Dec. 25, 2025

(54) Pain Treatment Apparatus and Methods

(71) Applicant: Evon Medics, LLC, Ellicott City, MD (US)

(72) Inventors: Evaristus Nwulia, Ellicott City, MD (US); Vamsi Reddy, Ellicott City, MD (US)

(21) Appl. No.: 18/753,722

(22) Filed: Jun. 25, 2024

(51) Int. Cl. A61M 21/02; A61M 11/02; A61M 16/06; A61M 16/14; A61M 16/20; A61M 21/00

(52) U.S. Cl. CPC A61M 21/02; A61M 11/02; A61M 16/0666; A61M 16/14; A61M 16/202; A61M 2021/0016; A61M 2021/0077

(57) ABSTRACT

In one aspect, a method of treating pain in a patient is provided. The method includes, in one phase, emitting each odorant of a plurality of odorants to a patient independently of other odorants of the plurality of odorants. And, upon emitting each odorant of the plurality of odorants, prompting the patient to perform a cognitive task.

### FIELD

This disclosure relates to pain treatment and, more specifically, an apparatus and methods for treating pain by olfactory stimulation.

### BACKGROUND

Many individuals have chronic pain, e.g., pain that has lasted for more than three months. Individuals with chronic pain may have limitations in mobility and the activities they can perform. Individuals with chronic pain also often experience anxiety and depression, which results from chronic pain causing a reduction in the prefrontal cortex (PFC) region of the brain and other deep brain connections critical to emotion, motivation, and cognitive functions. Chronic pain is commonly treated with pharmaceuticals including non-steroidal anti-inflammatory drugs (NSAIDS), opioids, topical analgesics, and adjuvants such as antidepressants and antiepileptic drugs. Treatment with such pharmaceuticals may have undesired side effects such as, for example, damage to other parts of the body (e.g., heart, kidney, etc.) and addiction (e.g., opioid dependence).

Chronic pain is often accompanied by a significant reorganization of the central nervous system activity and thus brain stimulation has been used to treat chronic pain. Examples of non-invasive brain stimulation approaches that have been used include repetitive transcranial magnetic stimulation (rTMS), transcranial direct current stimulation (tDCS), cranial electrotherapy stimulation, transcranial random noise stimulation, and reduced impedance and non-invasive cortical electrostimulation. These surface electrical and magnetic stimulation approaches can depolarize the dorsolateral prefrontal cortex (DLPFC), but they are limited in their ability to penetrate deep brain structures, such as the orbital and medial prefrontal cortex regions and medial temporal regions which play important roles in pain. Such brain stimulation approaches are also often expensive and generally not accessible for long-term daily treatment that is needed for long-term neuroplasticity. Indeed, such brain stimulation techniques have many drawbacks including varying patient responses, temporary pain relief, safety concerns, limited access, cost considerations, the need for customized approaches for each patient, the lack of home-based solutions, regulatory and ethical challenges, and uncertainties regarding long-term effects of such treatment.

Counter-irritation techniques, such as Transcutaneous Electrical Nerve Stimulation (TENS) of body extremities and application of topical capsaicin to local pain sites, have also been used to manage chronic pain. These counter-irritation techniques have several limitations including high costs, limited accessibility, undesired side effects, and that they only offer temporary pain relief.

### BRIEF DESCRIPTION OF THE DRAWINGS

FIG. 1A is a top perspective view of an odor delivery system.

FIG. 1B is a bottom view of an odor emitter of the odor delivery system of FIG. 1A.

FIG. 1C is a top view of an odor emitter of the odor delivery system of FIG. 1A.

FIG. 1D is a schematic diagram of the odor emitter of FIG. 1A.

FIG. 2A is a schematic diagram of the odor emitter of FIG. 1A showing control of air flow in the odor emitter.

FIG. 2B is a perspective view of an odorant cartridge of the odor emitter of FIG. 1A.

FIG. 3 is an example graphical user interface providing a patient with instructions to set up the odor delivery system of FIG. 1A for treatment.

FIG. 4A is a flow diagram of a method of operation of the odor delivery system of FIG. 1A in a first phase.

FIG. 4B is a flow diagram of a method of operation of the odor delivery system of FIG. 1A in a second phase.

FIG. 5 is an example graphical user interface prompting the user to perform a cognitive task.

FIG. 6 is a graph showing test results from pain treatment according to a short burst treatment method.

FIG. 7 is a graph showing test results from pain treatment according to a long burst treatment method.

FIG. 8A is graph showing the effect of no treatment, long burst treatment, and short burst treatment on pain severity.

FIG. 8B is a graph showing the effect of no treatment, long burst treatment, and short burst treatment on negative affect.

FIG. 9A is a graph showing the effect of no treatment, treatment without beta-caryophyllene (BCP), and treatment with 60% BCP on predicted pain level.

FIG. 9B is a graph showing the effect of no treatment, treatment without BCP, and treatment with 60% BCP on pain interference in daily activities.

FIG. 9C is a graph showing the effect of no treatment, treatment without BCP, and treatment with 60% BCP on negative affect.

FIG. 9D is a graph showing the effect of no treatment, treatment without BCP, and treatment with 60% BCP on duration of sleep.

FIG. 10 is a schematic diagram of a treatment system 100 including the odor emitter of FIG. 1D.

FIG. 11A is a perspective view of an odor cartridge according to another embodiment.

FIG. 11B is a bottom view of the odor cartridge of FIG. 11A.

### DETAILED DESCRIPTION

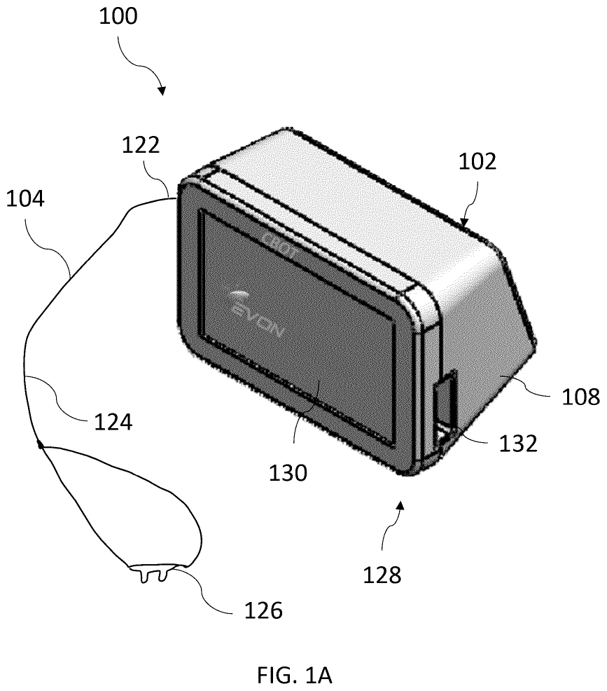

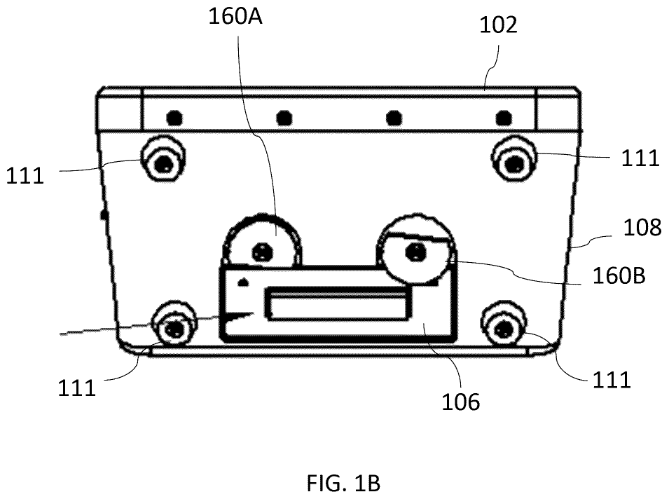

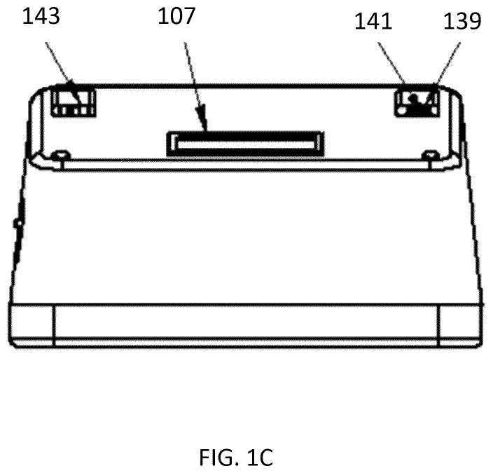

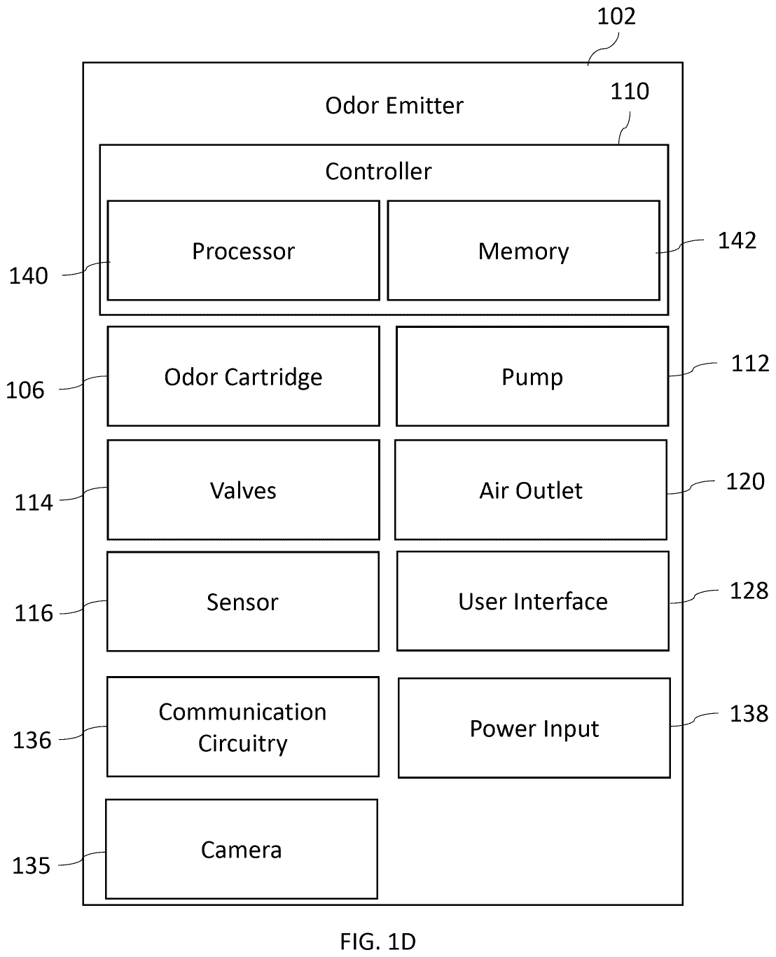

With respect to FIGS. 1A-1D, an odorant delivery system 100 is provided that includes an odor emitter 102 that may be used to treat pain (e.g., acute pain, chronic pain) in patients. The odorant delivery system 100 includes the odor emitter 102, odor delivery conduit such as a cannula 104, and an odorant source such as an odorant cartridge 106. In one form, the odorant delivery system 100 is a portable medical device that enables patients to take the odorant delivery system 100 with them to receive pain treatment outside of a clinic, for example, at their home.

The odor emitter 102 includes a housing 108 containing a controller 110, a gas source such as an airflow generator such as a pump 112, one or more valves 114, and one or more sensors 116. The gas source may be a device operable to move air or other breathable gases toward the patient during treatment as discussed below. While a pump 112 is described as the gas source in this discussion, in other forms the gas source may be, as examples, an airflow generator such as an air blower, pressurized air container, air compressor, and/or pressurized oxygen tank. The housing 108 may include a handle 107 that enables a user to carry and transport the odor emitter 102. The odor emitter 102 may include feet 111 mounted to the bottom of the housing 108 to stabilize the odor emitter 102 when positioned on a surface (e.g., a table). The housing 108 may receive the odorant cartridge 106 to connect the odorant cartridge 106 to the odor emitter 102. The odor emitter 102 is operable to emit odorants to the patient from the odorant cartridge 106.

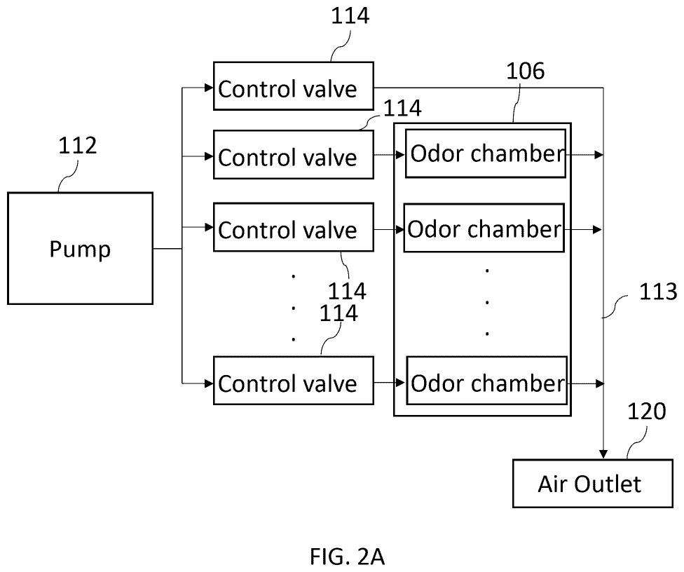

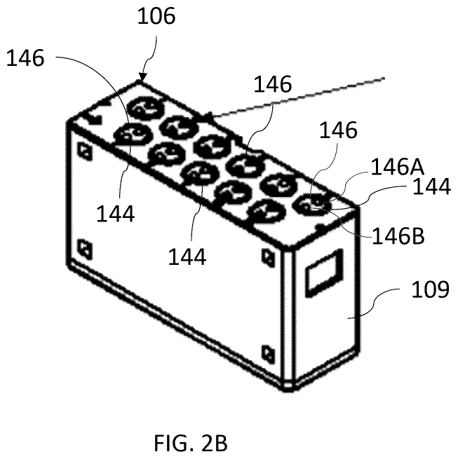

With reference also to FIG. 2A, the odor emitter 102 may include gas outlet such an air outlet 120 mounted on the housing 108 through which the odor emitter 102 emits air moved by the pump 112. The air outlet 120 may include a port to which an inlet end 122 of the cannula 104 may be connected. The odor emitter 102 may force air including odorant to flow out of the air outlet 120 to be provided to a patient to inhale. The air and/or odorant are forced from the air outlet 120 and along a tube 124 of the cannula 104 to nasal prongs 126 of the cannula 104 that may be received into a patient's nostrils during treatment. For example, the pump 112 may be operated to move air toward the air outlet 120 and the valve 114 associated with an odorant of the odorant cartridge 106 may be opened to permit odor to diffuse into the air flowing to the patient. The pump 112 may be operable to generate air flow in a range of four liters per minute to ten liters per minute. The odor emitter 102 may have a valve 114 corresponding to each odorant of the odorant cartridge 106 such that one valve 114 may be opened to release a desired odorant of the plurality of odorants of the odorant cartridge 106. The odor emitter 102 may also have a valve 114 that may be opened through which ambient or non-odorized air may be pumped to the patient (e.g., to clear the cannula of odorized air). The odor emitter 102 may include a convergence module 113 into which air and/or odorant flows upon opening of the valves 114. The pump 112 moves the air from the convergence module 113 to the air outlet 120.

The sensors 116 may be used to measure physiological parameters of a patient, such as heart rate, heart rate variability, electrocardiogram (ECG), pupil diameter/sizes, oxygen saturation, galvanic skin response, respiratory monitoring, temperature, etc. The sensor 116 may include a pulse oximeter, or photoplethysmography based sensor. The sensors 116 may measure the physiological parameters of the patient before, during, and/or after treatment via the odor emitter 102.

The odor emitter 102 may include a user interface 128 to facilitate interaction between a user, such as the patient, and the odor emitter 102. The user interface 128 may include a display screen 130 secured to the housing 108 to present information to the user. The display screen 130 may be a touchscreen display that the user may interact with (e.g., touch virtual buttons) to provide input to the odor emitter 102. The user interface 128 may also include physical buttons 132 that the user may press to control or provide input to the odor emitter 102. The user interface 128 may also include a speaker to audibly provide information to the user and/or a microphone to receive input (e.g., voice input) from the user. For example, the user may provide input in response to a prompt to perform a cognitive task, as discussed below. The odor emitter 102 may also receive input from a user via the user interface 128 that indicates the user's psychophysical symptoms and/or their subjective pain scores. For example, the odor emitter 102 may present a list of psychophysical symptoms (e.g., a questionnaire) via the user interface 128 and prompt the user to select which psychophysical symptoms they have from the list. Similarly, the odor emitter 102 may prompt the user to enter their subject pain scores via the user interface 128, e.g., each time the user receives treatment from the odor emitter 102. The odor emitter 102 may store data input by the user, e.g., to track treatment progress over time.

The odor emitter 102 may include communication circuitry 136 to communicate with another computing device. For example, the communication circuitry 136 may include a wired communication interface such as a USB port 143 and/or a wireless communication interface configured to communicate via Bluetooth, Wi-Fi, and/or cellular as examples. The odor emitter 102 may use the communication circuitry 136 to transfer data to another device. The communication circuitry 136 may communicate data relating to the patient and/or the treatment, for example, the measured physiological parameters of the patient, the input psychophysical symptoms of the patient, the subjective pain scores of the patient, data pertaining to administration of the treatment (e.g., treatment frequency), and patient inputs in response to cognitive task prompts (e.g., olfactory cognitive performance scores).

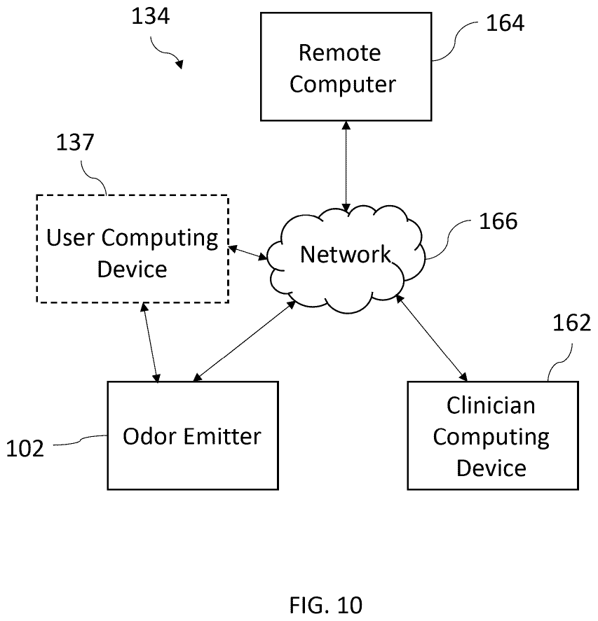

With respect to FIG. 10, the odor emitter 102 may be part of a treatment system 134 that includes a clinician computing device 162 and a remote computer 164 such as a server computer. The odor emitter 102, clinician computing device 162 and the remote computer 164 may communicate via a network 166, such as a Wi-Fi network, a cellular network, and/or the internet. The odor emitter 102 may communicate data relating to the patient and/or the treatment to the remote computer 164 for storage and/or analysis. The odor emitter 102 may communicate which odorants are in the cartridge 106 inserted into the odor emitter 102, which odorants are emitted, what order the odorants are emitted in, the amount of time each odorant is emitted for, the amount of time between the odorant emissions. The odor emitter 102 may also communicate physiological metrics or symptoms of the patient, for example, measured by the sensors 116 or input by the patient. The physiological metrics may be communicated with a time stamp indicating when the metrics were measured (e.g., before treatment, during treatment). The odor emitter 102 may also communicate assessment questions provided to the patient and patient responses, a session log, user responses to cognitive tasks, information about the odor emitter 102, error logs, and other data files. Where the odor emitter 102 is not connected to the network 166 (e.g., a Wi-Fi network and the internet), the odor emitter 102 may store the data until the odor emitter 102 is connected to the network 166, at which point the data is communicated to the remote computer 164 and/or clinician computing device 162. By storing data and uploading upon a connection to the network 166, the odorant delivery system 100 is able to be used by patients that may not have an internet connection at the home or a smartphone, thus increasing accessibility of the pain treatment. In some forms, the communication circuitry 136 of the odor emitter 102 is able to communicate via a cellular network to communicate with the remote computer 164 via the cellular network and/the internet. Such a configuration permits odorant emitters 102 to communicate data even when the patient does not have a home internet connection.

The clinician computing device 162 may receive the patient data and/or treatment data or access such data from the remote computer 164. The remote computer 164 may, for example, host a secure, HIPAA compliant web portal through which a clinician is able to access data associated with a patient's treatment. The clinician may access the treatment data, for example, when a clinician is analyzing the patient's treatment plan and progress. The clinician may update the patient's treatment plan using the clinician computing device 162 and send the updated plan to the remote computer 164 and/or odor emitter 102 for the patient's treatment. The clinician may, for example, customize the treatment plan for each patient based on their progress, e.g., adjusting the frequency of each treatment session, the length of each treatment session, the odorant emission duration, the interstimulus interval duration, the number of treatment cycles in a treatment session, the odor related cognitive tasks, and other parameters of the treatment method. The clinician is able to access patient treatment data in near real time and customize the treatment remotely from the patient. This permits the patient to receive modifications to their treatment plan without having to schedule an appointment with the clinician or bring their odor emitter 102 into a clinic to receive a modified treatment plan.

In some forms, the treatment system 134 may include a user computing device 137, such as a smartphone, that runs a computer application associated with the treatment system 134. The user computing device 137 may communicate with the odor emitter 102, for example, via Bluetooth, Wi-Fi, ethernet, and/or USB. The computer application of the user computing device 137 may prompt the user to perform cognitive tasks during treatment. The computer application may receive data from the odor emitter 102 and permit the user view data associated with their treatment, for example, data collected from each treatment session and aggregated results (e.g., such as pain severity over time). The computer application may permit the user to control the odor emitter 102 via the user computing device 137 instead of interfacing with the user interface 128 of the odor emitter 102. The computer application may thus be used as a virtual control device to operate the odor emitter 102. The computer application can interface with the odor emitter 102 directly and with the odor emitter 102, the remote computer 164, and clinician computing device 162 via the network 166. The computer application may permit patients, clinicians and other technical support users to communicate and control treatment via the odor emitter 102. The computer application may be configured to provide data visualization and/or access to raw data of data collected by the treatment system 134. The computer application may be configured to notify patients when their treatment plan is changed and provide clinicians with options to customize or change a current treatment plan. The computer application permits patients to contact a clinician with questions and/or to request changes to their treatment plan. The computer application permits clinicians to contact patients to get treatment progress and periodic (e.g., daily) updates. The computer application may be used as an interface to provide remote connectivity to the odor emitter 102 (e.g., with the remote computer 164) and may enable over the air firmware updates to the odor emitter 102. For example, the user computing device 137 may receive data (e.g., firmware updates) from the network 166 and the user computing device 137 may communicate such data to the odor emitter 102 over a direct, local connection such as Bluetooth, ethernet, or USB (e.g., to apply the firmware updates to the odor emitter 102).

The odor emitter 102 may include a camera 135. The odor emitter 102 may facilitate video conferencing between the patient and a clinician and use the camera 135 to capture images (e.g., a video feed) to send to the clinician over the network 166. The odor emitter 102 may capture sounds (e.g., speech) of the patient via the microphone of the user interface 128 and output sounds (e.g., speech) of the clinician via the speaker of the user interface 128 to facilitate communication between the patient and the clinician, e.g., when video conferencing. The odor emitter 102 may also permit the patient to have audio communication with the clinician without the use of the camera 135. The camera 135 may be used to capture images of the patient, for example, to monitor physiological parameters of the patient, such as pupil dilation. Use of the camera 135 may also permit clinicians to claim remote patient monitoring codes for billing for medical services.

The odor emitter 102 may include a power input 138 to receive electrical power to power the odor emitter 102. The power input 138 may be a port to which a power cord may be connected to receive power from a wall outlet. The power input 138 may also connect to a battery to receive electrical power from the battery. The odor emitter 102 may include a charging port 139 to which a power cord may be connected to charge the battery of the odor emitter 102. Inclusion of a battery in the odor emitter 102 to power the odor emitter 102 increases the portability of the odorant delivery system 100. The odor emitter 102 further includes a power switch 141 that may be used to switch the odor emitter 102 on (e.g., to use the odor emitter 102) or off (e.g., to conserve electrical power).

The controller 110 may include a processor 140 and a memory 142. The processor 140 may be configured to execute instructions stored in the memory 142 to operate the components of the odor emitter 102 as discussed herein, for example, to administer treatment, receive input from the user, and communicate information with a remote computing device (e.g., via the communication circuitry 136). The memory 142 may store programs and/or instructions for the processor 140 to execute to provide functionality to the odor emitter 102. The memory 142 also may be used to store sensed data and input from the patient in response to a prompt to perform a cognitive task. The processor 140 may include, as examples, a microprocessor, an application-specific integrated circuit (ASIC), or a field programmable gate array (FPGA). The memory 142 may include, as examples, random-access memory (RAM), read-only memory (ROM), electrically erasable programmable read-only memory (EEPROM), and flash memory.

With reference also to FIG. 2A, the processor 140 may be in communication with the pump 112, the valves 114, the sensor 116, the user interface 128, and the communication circuitry 136. The processor 140 may control the pump 112 to cause the pump 112 to move air toward the air outlet 120 and through the cannula 104 to the patient when the cannula 104 is connected to the air outlet 120. The pump 112 may be able to generate airflow with a flow rate in the range of 4 liters to 10 liters per minute. The flow rate of the pump 112 may be adjustable, for example, via a set screw on the pump 112 to increase or decrease the flow rate. In other forms, the flow rate of the pump 112 may be adjusted by the user through the user interface 128 and the processor 140 may adjust the speed of the pump 112 to provide the desired flow rate. The processor 140 is able to open and close the valves 114 to selectively release odorants from the odorant cartridge 106. The processor 140 may control the state of the valves 114 to emit air and/or odorant to the patient. To emit odorant, the processor 140 may operate the pump 112 and open the valve 114 associated with the odor chamber containing the odorant to be emitted. The air may pass through the odor chamber to odorize the air with the odorant of the odor chamber. The odorized air may then flow to the air outlet 120 and to the patient via the cannula 104. To emit non-odorized air (e.g., to clear the cannula 104 of odorized air), the processor 140 may open the valve 114 that is not associated with an odor chamber and pump the air to the air outlet 120.

With respect also to FIG. 2B, the odorant cartridge 106 includes a carrier 109 including a plurality of openings 144 to receive and support a plurality of odor chambers such as odor tanks 146. The odorant cartridge 106 is removable from the odor emitter 102 to swap out the odor tanks 146 with new odor tanks 146 as the odor tanks 146 are emptied. For example, the housing may include rotatable, asymmetric tabs 160A, 160B secured to the housing that are rotatable between a first configuration (see tab 160A) that permits the odorant cartridge to be inserted and withdrawn from the odor emitter 102 and a second configuration (see tab 160B) that inhibits insertion or removal of the odorant cartridge 106. To remove or withdraw the odorant cartridge 106 (e.g., to replace the odor tanks 146) the tabs 160A, 160B may be moved to the first configuration. To secure the odorant cartridge 106 in the odor emitter 102, the tabs 160A, 160B may be moved to the second configuration upon inserting the odorant cartridge 106. As one example, the odorant tanks 146 contain a volume of odorant in the range of about 2 milliliters to about 8 milliliters. As one specific example, the odorant tanks 146 have a volume of 3 milliliters.

Each odor tank 146 includes an inlet 146A and an outlet 146B. When the odor cartridge 106 is secured to the odor emitter 102, the inlet 146A and outlet 146B of each odor tank 146 are connected to air passages of the odor emitter 102. For example, the inlet 146A is connected to an air passage extending from the pump 112 and the outlet 146B is connected to an air passage extending to the air outlet 120. The inlet 146A and outlet 146B may form a fluid tight seal with the odor emitter 102 when secured thereto to inhibit fluid leakage through the connection therebetween. When the associated valve 114 is opened, the pump 112 may force air into the inlet 146A of the odor tank 146 and out the outlet 146B to odorize the air to be emitted to the patient. The odor tanks 146 may include threads that cooperate with threads of the openings 144 to secure the odor tanks 146 to the carrier 109.

Each odor tank 146 may contain an odor mixture having a different odorant profile or formulation such that each odor tank 146 provides a unique scent to the patient. The odor formulations may include essential oils of different scents, for example, banana oil, vanilla oil, lemon oil, cinnamon oil, rosemary oil, eucalyptus oil, ginger oil, mint oil, orange oil, citrus oil, clove oil, and lavender oil. Additional examples of scents that could be used include wintergreen, jasmine, roman/german Chamomile, basil, sage, thyme, sandalwood oil, lemon grass, frankincense, bergomot, green apple, helichrysum, pelargonium graveolens flower oil, libanum carteri resin oil, helichrysum angustifolium oil, rosa damascena flower extract, artemesia pallens flower oil, Lavandula angustifolia oil, santalum album wood oil, picea mariana oil, valeriana officinalis root oil, marjoram oil, juniper oil, black pepper oil, copaiba oil, vetiver oil, cedarwood oil, ylang ylang oil, turmeric oil, cardamom oil, fennel oil, hyssop oil, birch oil, black seed oil (nigella sativa), cajeput oil, caraway oil, spearmint oil, nutmeg oil, anise oil, bay leaf oil, camphor oil, cypress oil, tea tree oil, myrrh oil, neroli oil, sweet birch oil, petitgrain oil, tarragon oil, galbanum oil, mugwort oil, spikenard oil, blue tansy oil, costus root oil, elemi oil, fir needle oil, mandarin oil, litsea cubeba oil, sweet orange oil, grapefruit oil, and tangerine oil. Each odor mixture is comprised of many different chemicals (e.g., 10 to 100) and ratios of chemicals that provide a unique scent to a patient. The odor formulations may include an essential oil diluted with a solvent, for example, 1:1 ratio by volume of essential oil to solvent. The solvent may be, as examples, propylene glycol, polysorbate 20, polysorbate 60, and polysorbate 80. In some forms, the odorants may be mixed together to create an odorant combination from two or more unique odorants (e.g., banana and vanilla) which may have a cumulative effect on the activation of brain regions.

The odor formulations of some or all of the odor tanks 146 may be mixed with beta-caryophyllene (BCP) which may enhance the effect of the pain treatment, as discussed below. BCP may be added to the odor formulations to increase the BCP content beyond any natural levels of BCP content in the odors, if any. The final odorant mixtures may, for example, contain a BCP content in the range of about 5% to about 80% by volume. As a more specific example, the odorant mixtures may have a BCP content of about 20% to about 70%, for instance, 30% or 60% by volume. The BCP content added to the essential oil odor formulations may be a BCP mixture comprising a clove oil mixture mixed with a diluted BCP mixture. The clove oil mixture may include clove oil diluted with a solvent (e.g., propylene glycol), for example, a 1:1 ratio by volume of clove oil to solvent. The diluted BCP mixture may include BCP mixed with a solvent (e.g., propylene glycol), for example, a 3:2 ratio by volume of BCP to solvent. The final BCP mixture is formed by mixing the clove oil mixture and diluted BCP mixture together, for example, in a 1:1 ratio by volume. Some odorants such as essential oils naturally contain BCP and thus the amount of BCP content added to the essential oil may be adjusted to account for the odorants natural BCP content.

In other forms, the odor emitter 102 has a chamber that receives odor tanks 146 therein. The odor tanks 146 may have larger volumes than the odor tanks of the odorant cartridge 106 for prolonged use of the odor emitter 102.

Each valve 114 may be associated with one of the odor tanks 146 of the odorant cartridge 106. The processor 140 may open and close the valves 114 to permit odor of the odor tanks 146 to nebulize and diffuse into the airflow generated by the pump 112. The processor 140 may open one valve 114 at a time to permit only one odorant to be output to the patient. The valves 114 may be normally closed valves 114 that may be actuated by the processor 140 to open and may automatically return to their closed position upon the processor 140 ceasing to actuate the valve 114.

The processor 140 may output signals and information to the patient via the user interface 128 to administer treatment and may receive input from the patient via the user interface 128 to operate the odor emitter 102 and receive responses to treatment prompts. The processor 140 may also collect physiological information of the patient via the sensors 116 and may store the collected data in memory 142. The processor 140 may use the communication circuitry 136 to transfer collected data to a remote computer, for example, a clinician computing device 162.

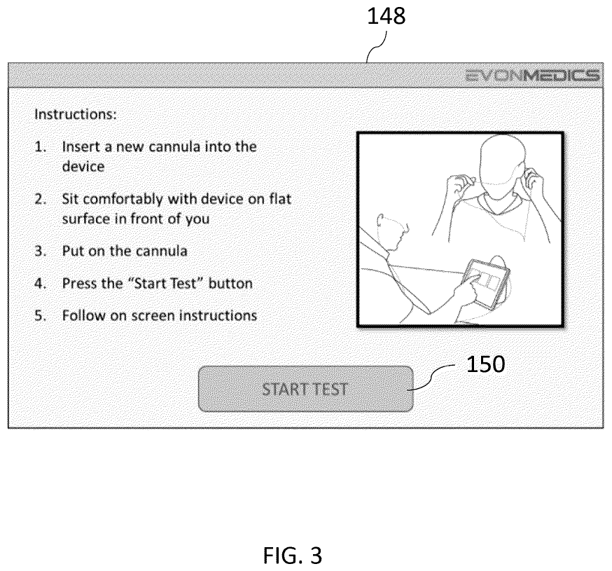

With respect to FIGS. 3-5, the odor emitter 102 may perform a non-invasive method that has been found to provide long term pain treatment in patients as discussed herein. The processor 140 may present a graphical user interface screen 148 on the display screen 130 providing the patient with instructions for beginning pain treatment with the odor emitter 102. As shown, the odor emitter 102 may prompt the patient to connect a new cannula 104 to the air outlet 120. The patient may insert the inlet end 122 of the cannula 104 into the air outlet 120. The odor emitter 102 may indicate that the odor emitter 102 should be placed on a flat surface in front of the patient. The odor emitter 102 may prompt the patient to put on the cannula 104, for example, to wear the cannula 104 such that the nasal prongs 126 extend into the patient's nostrils so that air emitted by the odor emitter via the air outlet 120 flows into the patient's nose for inhalation. The odor emitter 102 may prompt the user to press a virtual button 150 displayed on the display screen 130 to start the treatment when they are ready.

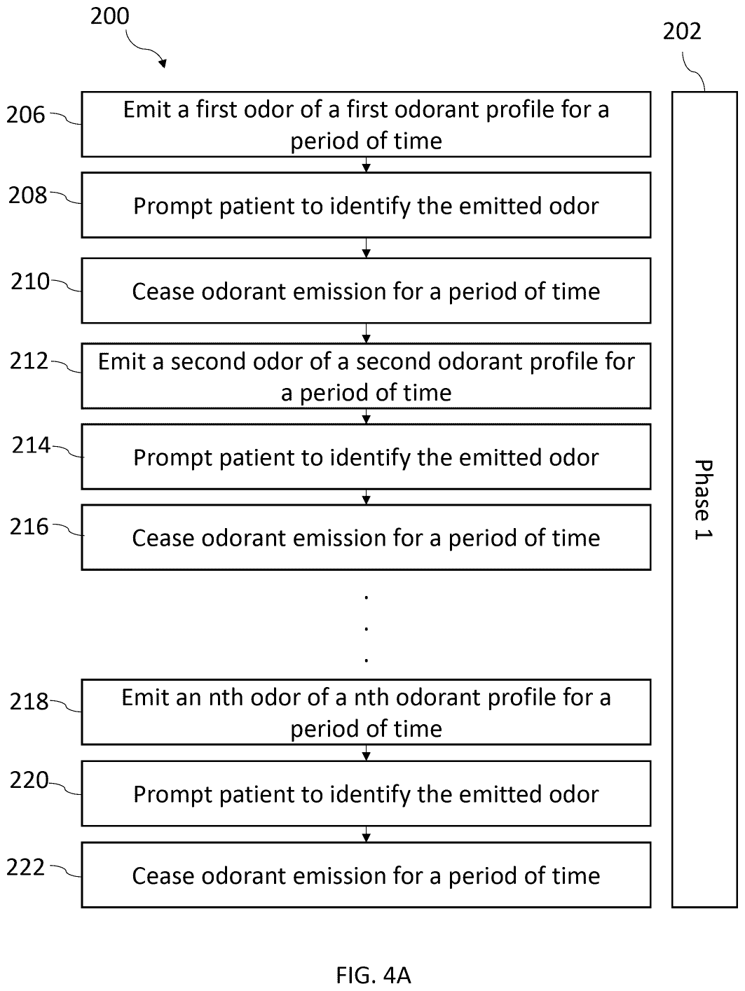

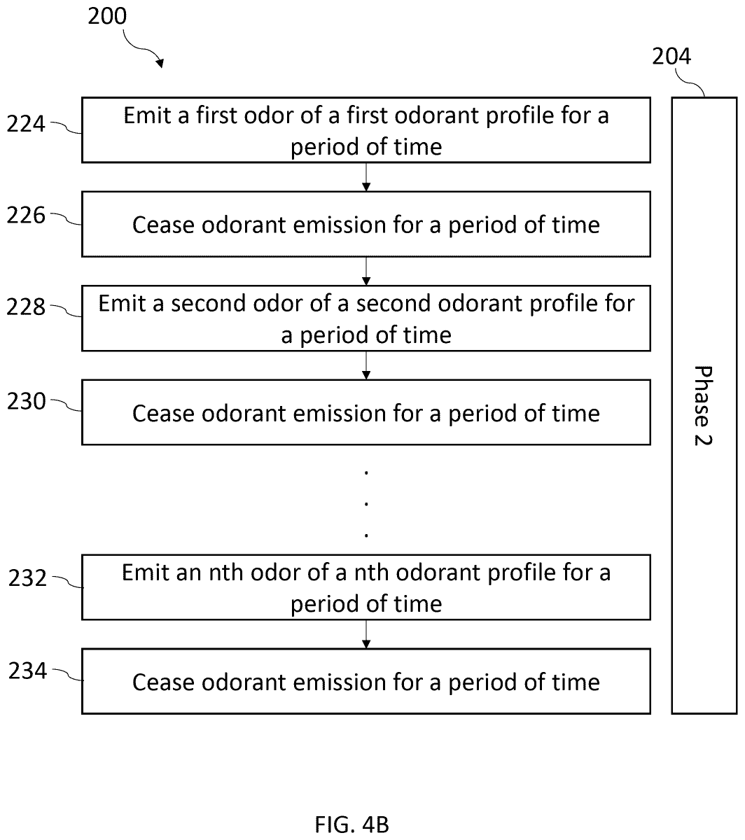

Upon the processor 140 receiving input from the patient selecting the virtual button 150 to start the treatment, the processor 140 may begin operating components of the odor emitter 102 to provide the odorants to the patient according to a treatment method 200. The treatment method 200 may include two treatment phases: Phase One; and Phase Two. The steps of Phase One 202 are shown in FIG. 4A and the steps of Phase Two 204 are shown in FIG. 4B. In some forms, the treatment method 200 includes four treatment phases: Phase One; Phase Two; Phase Three; and Phase Four. The steps of Phase Three may be the same as those of Phase One and the steps of Phase Four may be the same as those of Phase Two and thus the discussion of Phase Three and Phase Four will not be repeated for conciseness and clarity.

With respect to FIG. 4A, in Phase One 202 of the treatment method 200, once the patient has provided input to begin the treatment, the odor emitter 102 may emit 206 a first odor of a first odorant profile for a period of time. The first odor may be an odor of one of the plurality of odor tanks 146 discussed above that each have a unique odorant profile. To emit the first odor, processor 140 may cause the pump 112 to operate and move air out the air outlet 120 and through the cannula 104 to the patient. The processor 140 may open a first valve 114 associated with a first odor tank 146 having the first odor to permit odorant of the first odor tank 146 to diffuse into the air flowing to the patient. The valves 114 associated with the other odor tanks 146 may be closed to inhibit odorant of the other odor tanks 146 from flowing to the patient such that only one odorant is administered to the patient at time. As one example, the first odor may be emitted for a period of time in the range of about 5 seconds to about seventy seconds. In one specific example, the odorant is emitted for nine seconds. In one specific example, the odorant is emitted for 15 seconds. In another specific example, the odorant is emitted for 60 seconds. As the patient smells the odor, the olfactory brain regions of the patient are stimulated (e.g., the primary olfactory cortex, orbitofrontal cortex, and secondary olfactory cortex).

Upon emitting the first odor, the odor emitter 102 prompts 208 the patient to perform a cognitive olfactory psychophysical training task (referred to as a cognitive task) that stimulates the olfactory cortex, orbitofrontal cortex, and medial temporal regions of the brain of the patient. The cognitive task is a task where the patient uses conscious thought to process information about the scents they smell. The cognitive task activates the same regions of the brain that are activated when the patient smells the emitted odorant. Activating both the olfactory cortex, orbitofrontal cortex, and medial temporal regions of the brain provides a synergistic effect to cause long-term neuroplastic changes in the brain that inhibit chronic pain induced shrinkage of such regions of the brain. In this example, the odor emitter 102 prompts the patient to identify the emitted odor as the cognitive olfactory psychophysical training task.

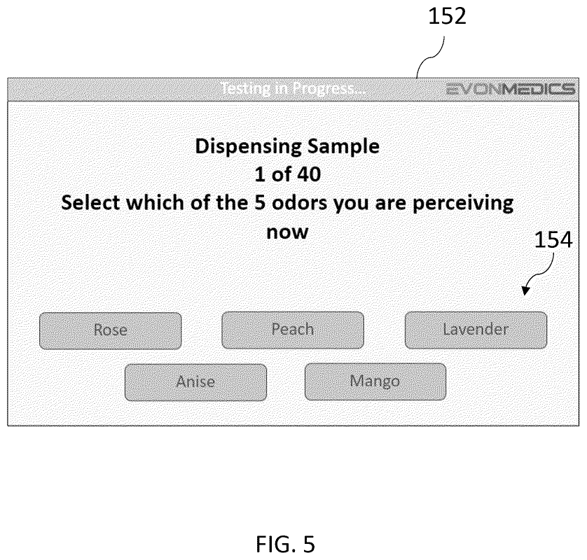

With respect to FIG. 5, the odor emitter 102 may present a graphical user interface screen 152 via the display screen 130 prompting the user to identify the emitted odor. In the example shown in FIG. 5, the odor emitter 102 displays five options 154 of odors that the user may select. The patient may input their selection of one of the options 154, for example, by touching the virtual button on the display screen 130 associated with the option 154. In some forms, the patient may also use the physical buttons 132 of the odor emitter or voice input to provide their selection of an option 154. The odor emitter 102 may prompt the patient to select an option 154 within a period of time, for example, within about three seconds to about twenty seconds of displaying the prompt. In one specific example, the patient is prompted to make a selection within five seconds of displaying the prompt.

Other examples of cognitive tasks that could similarly be used to effect neuroplastic changes to the brain include odor discrimination tasks, odor memory tasks, and odor threshold tasks. These cognitive tasks may be performed as alternatives to or in addition to the odor identification task described above. These tasks may also be separate treatment methods from method 200 and performed apart from method 200 (e.g., as an alternative treatment method altogether).

Where odor discrimination is used, the odor emitter 102 prompts the user to identify whether two scents output by the odor emitter 102 are the same or different. For example, the odor emitter 102 may inform the patient that it will emit a first scent and subsequently emit a second scent and prompt the patient to identify whether the first scent is the same as the second scent or whether the patient smelled nothing at all. As one example, the first scent may be emitted for two to five seconds, the odor emitter 102 may clear the line by emitting air for two to five seconds, and then emit the second scent for two to five seconds. The odor emitter may repeat this process several times (e.g., four times) to prompt the user to make several scent comparisons. The accuracy of patient responses may be tracked for accuracy.

Where odor memory is used, the odor emitter 102 may emit one or more odors after completing Phase One 202 of the treatment and asking the patient to identify whether the emitted odor was previously emitted to the patient during the treatment. Where odor memory is used, Phase One 202 may be modified to remove the steps of prompting the patient to identify the odors upon emission of each odor. The accuracy of patient responses may be tracked for accuracy.

Where odor thresholds are used, the odor emitter 102 may emit two odors in succession and prompt the user to identify which odorant emission had the stronger smell. For example, the odor emitter 102 may inform the patient that it will emit a first scent and subsequently emit a second scent and then prompt the patient to identify whether the first scent or second scent smelled stronger. As one example, the first scent may be emitted for two to five seconds, the odor emitter 102 may clear the line by emitting air for ten seconds, and then emit the second scent for two to five seconds. The odor emitter may repeat this process several times (e.g., sixteen times) to prompt the user to make several scent comparisons. In some forms, one of the first scent and the second scent is air without an odorant. In some forms, one of the first scent and second scent emitted from the odor emitter 102 includes a concentration of N-butanol. In some forms, in each scent comparison cycle, one of the scents is air and the other is a varying concentration of N-butanol (e.g., the concentration increasing with each cycle or random). The scents may be emitted in a random order to inhibit the patient from relying on their memory (e.g., whether the air or N-butanol is emitted first). The accuracy of patient responses may be tracked for accuracy.

The odor emitter 102 may cease 210 odorant emission for a period of time (referred to as the inter-stimulus interval) after the period of time for emitting the first odor has expired. To cease odorant emission, the processor 140 may close all of the valves 114 of the odor tanks 146 so that odorant is not being emitted from any of the odor tanks 146. The pump 112 may continue to operate to flow air through the cannula 104 to clear the odor emitter 102 and cannula of odorant. In this example, inter-stimulus interval is a time period in the range of about 25 seconds to about 65 seconds. In one specific example, the inter-stimulus interval is 30 seconds. In another specific example, the inter-stimulus interval is 60 seconds. In some forms, the odor emitter 102 prompts 208 the patient to perform the cognitive task upon ceasing odorant emission.

Upon expiration of the period of time for ceasing odorant emission, the odor emitter 102 emits 212 a second odor having a second odorant profile for a period of time. The second odor is different from the first odor, having a unique odorant profile. To emit the second odor, the processor 140 may operate the pump 112 and valves 114 similar to emission of the first odor described above, but opening a second valve 114 associated with a second odor tank 146 having the second odor to permit odorant of the second odor tank 146 to diffuse into the air flowing to the patient. The valves 114 associated with the other odor tanks 146 may be closed to inhibit odorant of the other odor tanks 146 from flowing to the patient such that only one odorant is administered to the patient. The second odor may be emitted for the same length of time as the first odor.

The odor emitter 102 prompts 214 the patient to perform a cognitive task and ceases 216 odorant emission for a period of time as described above with regard to the first odor. In this example, the patient is prompted to identify the second odor based on a set of options displayed on the display screen 130 similar to that shown, for example, in FIG. 5.

The odor emitter 102 may continue to repeat the above process n times to emit a set of n number of unique odors each having different odorant profiles (e.g., a chemical composition that provides different scents such as banana, vanilla, lemon, cinnamon, rosemary, eucalyptus, ginger oil, mint oil, orange oil, citrus oil, clove oil, and lavender oil). For example, the odorant cartridge 106 may hold ten odor tanks 146 each having a unique odorant. The odor emitter 102 may repeat the steps of emitting 218 the odorant, prompting 220 the user to perform the cognitive task (such as identifying the odor), and ceasing 222 odorant emission n times until each unique odor of the odorant cartridge 106 has been emitted to the user once. The odors may be emitted to the patient in a random order generated by the processor 140 for each treatment cycle. Emission of the odorants in a random order ensures that the patient is not able to rely on their memory when identifying odorants. In some forms, the odor emitter 102 selects a subset of odors of the odorant cartridge 106 to emit to the user in Phase One 202. For instance, where the odorant cartridge 106 has ten odor tanks 146, the odor emitter 102 may select to emit a subset of the odorants, for example six odors in Phase One 202.

With respect to FIG. 4B, in Phase Two 204 of the treatment method 200, the odor emitter 102 again emits each of the set of unique odorants to the patient one at time, but without prompting the patient to perform a cognitive task. In Phase Two 204, the odor emitter 102 emits 224 a first odor of a first odorant profile for a period of time similar to step 206 described above. Upon expiration of the period of time, the odor emitter 102 ceases 226 odorant emission for a period of time similar to step 208 described above. The first odor of Phase Two 204 may be the same odor or a different odor than the first odor emitted in Phase One 202. For example, the order of the odorants emitted in Phase Two 204 may again be randomly generated. After the period of cessation has elapsed, the odor emitter 102 may emit 228 a second odor of a second odorant profile for a period of time and then cease 230 odorant emission for a period of time. The steps of emitting 232 an odorant and ceasing 234 odorant emission may be repeated n times until each odorant of the set of n odorants has been emitted.

Upon completion of Phase Two 204, the odor emitter 102 may continue on to Phase Three, which has steps similar to those of Phase One 202 of FIG. 4A discussed above. Upon completion of Phase Three, the odor emitter 102 may continue on to Phase Four, which has steps similar to those of Phase Two 204 of FIG. 4B discussed above. The repetitive stimulation of the brain with the odorants according to these intervals (e.g., emission for period of time and cessation for a period of time) maintains persistent activation of the olfactory regions of the brain without habituation. The treatment method sustains activation of the olfactory regions of the brain over the course of the treatment session (e.g., for 60 minutes) without signification reduction in activation. The odorants may be fortified with a selective endocannabinoid-2 receptor agonist, such as BCP, which has strong anti-inflammatory and antinociceptive effects and increases the activation of the olfactory cortex and medial temporal regions of the brain. The cognitive olfactory psychophysical training tasks, such as the patient identifying the type of odor, activates the olfactory cortex and medial temporal regions of the brain that, with the stimulation from the odorants, effects long-term neuroplastic changes to the brain that inhibits chronic pain induced brain shrinkage.

The treatment method may be administered daily to continue to effect neuroplastic changes in the brain associated with chronic pain. The neuroplastic changes may take place over time and reduce pain experienced by the patient by forming and reorganizing synaptic connections in the brain caused by stimulation of the olfactory cortex region of the brain using scents and the cognitive tasks performed by the patient. For example, this treatment alters the connections (functional connectivity) between olfactory brain regions and brain regions involved in pain perception and pain control, including the thalamus, periaqueductal gray matter (PAG), insular cortex, and medial temporal lobe (MTL) ROIs including amygdala, hippocampus, and parahippocampus.

While the odor emitter 102 may perform a method as described above, the odor emission time and the inter-stimulus interval time may be adjusted. For example, a clinician may adjust the odor emission time, inter-stimulus interval time, frequency of acquisition of symptoms and subjective pain scores, duration of overall session, the number of stimulation cycles per session, the frequency of sessions per week to customize the treatment for each patient. Adjustments may also be made based on the patient's progress in pain management. The clinician may adjust the settings of the odor emitter 102 using the user interface 128 of the odor emitter 102 or may adjust the settings remotely for example, by communicating with the odor emitter 102 remotely (e.g., via Bluetooth or network 166) using the clinician computing device 162.

In other approaches, the odor emitter 102 may emit a combination of odorants together during one or all of the phases. For example, the odor emitter 102 may open two or more valves 114 associated with different odorants to emit a combination of odorants to the user.

In some forms, the odorant cartridge 106 has a plurality of odorant tanks 146 that each contain different odorants and one or more tanks that contains BCP. When emitting an odorant during treatment, the odor emitter 102 may open a valve 114 associated with an odorant to be emitted and a valve 114 associated with the BCP tank to emit the odorant and BCP together. BCP may be emitted with each odorant or a limited subset of odorants, such as those that do not naturally contain BCP. Having a separate BCP tank may permit standard odorants (such as essential oils) to be used by the odor emitter 102, with BCP being mixed with the odorant during emission rather than being pre-mixed into the odorant tanks 146. The odor emitter 102 may control the amount of BCP emitted with each odorant such that the amount of BCP content is able to be adjusted, e.g., by a clinician to customize the patient's treatment.

To test the effectiveness of the odor emitter 102 and which treatment parameters are most effective, testing was conducted to compare activation of the medial olfactory cortex (including anteromedial or posterior medial regions) when each odor is emitted in a short burst mode or a long burst mode. In the short burst mode, the treatment was administered to a patient in four phases as discussed above with odorant being emitted for a period of nine seconds followed by a 30 second interstimulus interval. In the long burst mode, the treatment was administered to a patient in two phases as discussed above with odorant being emitted for a period of 60 seconds followed by a 60 second interstimulus interval.

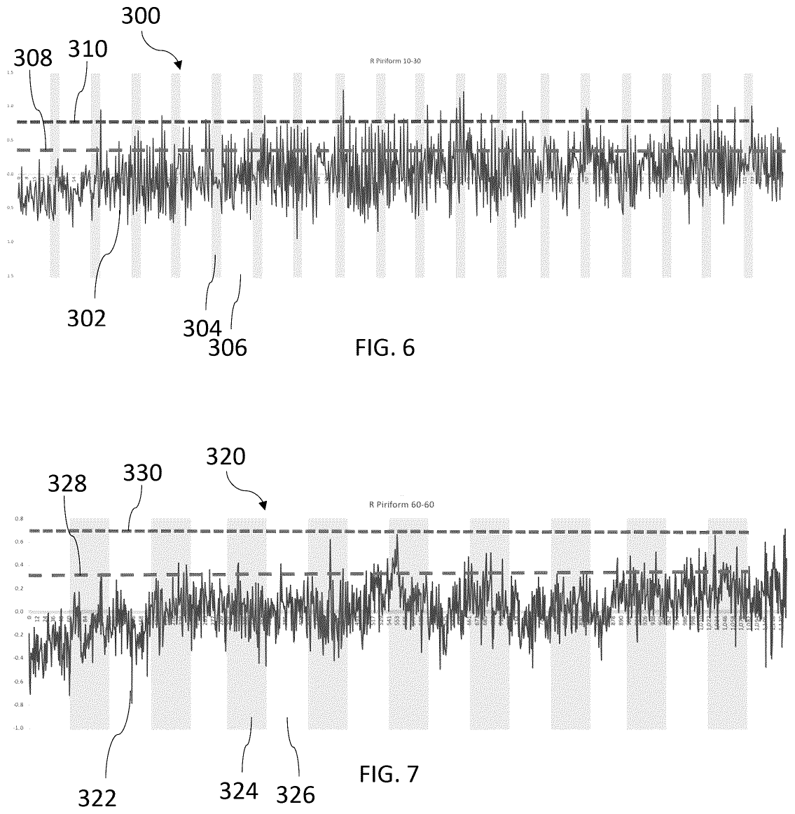

With respect to FIG. 6, a graph 300 is provided showing activation of the olfactory cortex over the course of a treatment session according to treatment method 200 using the short burst mode, where the odor emitter 102 emitted each odor separately in nine seconds intervals followed by a 30 second inter-stimulus interval between emission of each odor. In the graph 300, the X-axis is time and the Y-axis is the activation of the olfactory cortex as measured by functional magnetic resonance imaging (fMRI). The line 302 represents the measured activation of the olfactory cortex over time. The shaded bars 304 on the graph indicate when the odor emitter 102 was emitting an odorant to the patient. The unshaded regions 306 adjacent the shaded bars 304 indicate inter-stimulus intervals or times when the odor emitter 102 was not emitting an odorant to the patient. The line 308 indicates olfactory cortex activation of 3% and the line 310 indicates olfactory activation of 7%. As shown, in the short burst mode, activation of the olfactory cortex achieved or exceeded 3% peak activation by emission of the second odorant and many odor emission cycles achieved or exceeded a 7% peak activation.

With respect to FIG. 7, a graph 320 is provided that is similar to the graph 300 of FIG. 6. The graph 320 shows activation of the olfactory cortex over the course of a treatment session according to the treatment method 200 using the long burst mode, where the odor emitter 102 emitted each odor separately in 60 second intervals, followed by a 60 second inter-stimulus interval between emission of each odor. In graph 320, the X-axis is time and the Y-axis is the activation of the olfactory cortex as measured by functional magnetic resonance imaging (fMRI). The line 322 represents the measured activation of the olfactory cortex over time. The shaded bars 324 on the graph indicate when the odor emitter 102 was emitting an odorant to the patient. The unshaded regions 326 adjacent the shaded bars 324 indicate inter-stimulus intervals or times when the odor emitter 102 was not emitting an odorant to the patient. The line 328 indicates olfactory cortex activation of 3% and the line 330 indicates olfactory activation of 7%. As shown, in the long burst mode, activation of the olfactory cortex achieved or exceeded 3% peak activation during several odorant emission cycles, but none of the odor emission cycles achieved or exceeded a 7% peak activation.

Testing was further conducted to evaluate whether emission of odor in the short burst mode or the long burst mode as described above more effectively alters functional connectivity between medial temporal affective network or other networks involved in pain processing after seven days of daily olfactory stimulations. The testing involved providing seven days of treatment to patients with chronic back pain. The test results, a subset of which are shown in Table 1 below, show that the baseline olfactory activations were associated with prominent changes in the functional connectivity between certain regions of the brain relevant to pain. In Table 1, the coefficient value is a coefficient of the association between the olfactory activation and the functional connectivity between regions of the brain after seven days of treatment. The Robust SE values are the robust standard error associated with the coefficient. The “t” values are the t-statistic associated with the coefficient. The P values are the statistical significance associated with the coefficient. Meta-analysis of resting state functional connectivity of chronic pain shows that the medial temporal lobe (MTL) plays a role in nociception and that increased resting state connectivity involving the MTL is associated with antinociceptive effects of successful pain treatment.

TABLE 1
Association of Baseline Olfactory Activations
and 7-Day Functional Connectivity

| Olfactory | Associated | | | | |
|---|---|---|---|---|---|
| regional | functional | Coeffi- | Robust | | P |
| activations | connectivity | cient | SE | t | value |
| Left | R. Piriform-L. Piriform | 0.44 | 0.09 | 4.76 | <0.0001 |
| Piriform | L. Piriform-R. MTL- | 0.38 | 0.11 | 3.32 | 0.003 |
| | subPiriform | | | | |
| Right | R. anterior OFC-PAG | −0.148 | 0.06 | −2.35 | 0.027 |
| Piriform | R. Piriform-L. Piriform | 0.42 | 0.13 | 3.26 | 0.003 |

The test results also showed (see Table 3, below) that use of the treatment method 200 using the short burst mode significantly reduced the functional connectivity (post-treatment vs. pre-treatment) between the periaqueductal gray (PAG) and right anterior cingulate cortex (rACC), which may help dampen the affective component of pain in patients.

Analysis was also conducted to evaluate pre-treatment and post-treatment functional connectivity of the PAG to evaluate whether these treatment methods provide analgesia through reduced PAG to OFC connectivity. Specifically, linear mixed effect models were used to examine associations between pre-treatment to post-treatment changes in resting state functional connectivity of candidate networks with changes in pain intensities. The results are shown in Table 2 below. As shown in Table 2, a unit increase in the functional connectivity between the periaqueductal gray matter and the right anterior cingulate cortex (PAG-rACC) was associated with a mean increase of pain by 5.57 units. Also in Table 2, a unit increase in functional connectivity between L. Piriform-R. Piriform and R. Piriform-L. Piriform were significantly associated with reductions in pain severity (P=0.004 and P=0.012, respectively). The results indicate that the most significant networks associated with pain reduction are the right piriform to right medial temporal lobe (MTL) to subpiriform network. Referring to Table 1 above, the test results show that increased baseline activation of the piriform by short stimuli predicted increased functional connectivity in the right piriform to right MTL to subpiriform network.

TABLE 2
Effect of Unit Change in Network Functional
Connectivity on the Mean Level of Pain

| Networks | Coefficient | SE | P |
|---|---|---|---|
| PAG-rACC | 5.57 | 2.55 | 0.029 |
| L. Piriform-R. Piriform | −4.39 | 1.52 | 0.004 |
| L. Piriform-R. anterior OFC | −7.02 | 2.33 | 0.003 |
| R. MTL-subPiriform-L. MTL-subPiriform | −4.00 | 1.98 | 0.043 |
| R. Piriform-L. MTL-subPiriform | −4.69 | 2.25 | 0.037 |
| R. Piriform-R. MTL-subPiriform | −7.35 | 2.03 | 0.000 |
| L. Piriform-R. MTL-subPiriform | −5.95 | 2.37 | 0.012 |
| R. Piriform-L. Piriform | −5.06 | 2.01 | 0.012 |
| R. Piriform-L. MTL-subPiriform | −5.97 | 2.06 | 0.004 |
| R. Piriform-R. MTL-subPiriform | −7.15 | 1.09 | <0.0001 |
| L. Piriform-L. MTL-subPiriform | −4.16 | 1.91 | 0.03 |

In Table 2, the coefficient value is a coefficient of the association between pre-treatment to post-treatment changes in resting state functional connectivity of candidate networks with changes in pain intensities. The SE values are the robust standard error associated with the coefficient. The P values are the statistical significance associated with the coefficient.

With continued reference to Table 2, there was a significant association between increases in functional connectivity between L. Piriform-R. Piriform and R. Piriform-L. Piriform and reductions in pain severity (P=0.004 and P=0.012, respectively), which is consistent with other studies indicating the most significant networks associated with pain reduction are the right piriform to right MTL to subpiriform network.

TABLE 3
Effect of Short versus Long Burst CBOT on 7-
Day Change in Network Functional Connectivity

| | Coefficient | SE | P values |
|---|---|---|---|
| R. ACC-Thalamus | | | |
| long burst | .1438012 | .0516759 | 0.005 |
| Short burst | .0169795 | .0438664 | 0.699 |
| PAG-R. lateral OFC | | | |
| long burst | .0947808 | .0399973 | 0.018 |
| Short burst | −.0343094 | .0382009 | 0.369 |
| PAG-R. ACC | | | |
| long burst | −.0268655 | .0430153 | 0.532 |
| Short burst | −.0848238 | .0366708 | 0.021 |
| L. posterior OFC-R. | | | |
| lateral OFC | | | |
| long burst | −.0823236 | .0361838 | 0.023 |
| Short burst | .0098422 | .0396177 | 0.804 |
| R. Piriform-R. | | | |
| lateral OFC | | | |
| long burst | .0264109 | .0393521 | 0.532 |
| Short burst | .0602665 | .034481 | 0.08 |
| L. Piriform-R. Piriform | | | |
| long burst | .0517258 | .0527066 | 0.326 |
| Short burst | .091405 | .0423262 | 0.031 |

In Table 3, the coefficient value is a coefficient of the association between long burst treatment or short burst treatment with functional connectivity of candidate networks. The SE values are the robust standard error associated with the coefficient. The P values are the statistical significance associated with the coefficient.

The conclusion of the above testing is that both short burst and long burst treatment paradigms increased activation of the olfactory cortex by 3% or more, but that the short burst paradigm is more effective in activating the olfactory cortex over repeated cycles of stimulations compared to the long burst stimuli. The test results also show that short burst treatment paradigm results in more significant (P<0.05) change in functional connectivity between the medial temporal affective or pain-related networks (networks involving the meta-analysis validated MTL regions), but not necessarily the OFC region. Moreover, the test results show that the short burst treatment paradigm reduced the functional connectivity between the PAG and ACC, which indicates reduced engagement of an affective pain network node by short burst treatment. The test results also confirm the involvement of the PAG in the antinociceptive effect of the treatment but highlight the MTL regions as better indices of the antinociceptive effects of the treatment. In short, the test results indicate that treatment using the short burst mode is more effective in treating chronic pain.

Additionally, the test results show that both short burst treatment and long burst treatment led to substantial improvements in pain intensity, with short burst treatment showing the most improvement. For example, by the seventh day of treatment, nine out of fourteen (64.3%) recipients of short burst treatment had experienced a 30% reduction in pain whereas six out of fourteen (42.9%) recipients of long burst treatment experienced 30% reduction in pain; and by the fourteenth day of treatment, eleven out of fourteen (78.6%) recipients of short burst treatment had experienced a 30% reduction in pain whereas nine out of fourteen (64.3%) recipients of long burst treatment experienced 30% reduction in pain. Because the mean change in pain and negative affect were more profound for short burst treatment, the difference in treatment response was even more profound when using 50% reduction in pain. Specifically, by the fourteenth day of treatment, ten out of fourteen (71.4%) recipients of short burst treatment had a 50% reduction in pain whereas five out of fourteen (37.5%) recipients of long burst treatment had a 50% reduction in pain.

To estimate the average treatment effect (ATE) and the potential-outcome means (POMs), a ‘telasso’ (with log odds) function in the Stata 18 statistical package was used and the results are included in Table 4 below. This function uses augmented inverse probability weighting (AIPW) for parameter estimation, while using lasso methods to select from potential control variables to be included in the model.

TABLE 4
Average Treatment Effects of Short Burst vs. Long
Burst from Treatment Effects Analysis with Lasso

| | | Coeffi- | Robust | P |
|---|---|---|---|---|
| Outcome | Intervention | cient | SE | value |
| 50% Reduction: | ATE: Short vs. long | 0.41 | 0.17 | 0.017 |
| Pain | burst | | | |
| | POM: Long burst vs. | 0.33 | 0.13 | 0.009 |
| | none | | | |
| 50% Reduction: | ATE: Short vs. long | 0.40 | 0.15 | 0.006 |
| Negative | burst | | | |
| affect | POM: Long burst vs. | 0.29 | 0.10 | <0.0001 |
| | none | | | |

The results of Table 4 indicate that the log odds of having a 50% pain reduction by the fourteenth day of short burst treatment would be 0.41 units more than with long burst treatment. And the log odds of having a 50% pain reduction by the fourteenth day of long burst treatment are 0.33 log odds more than with no treatment. Similarly, the ATE for 50% reduction in negative affect is significant comparing short burst treatment to long burst treatment.

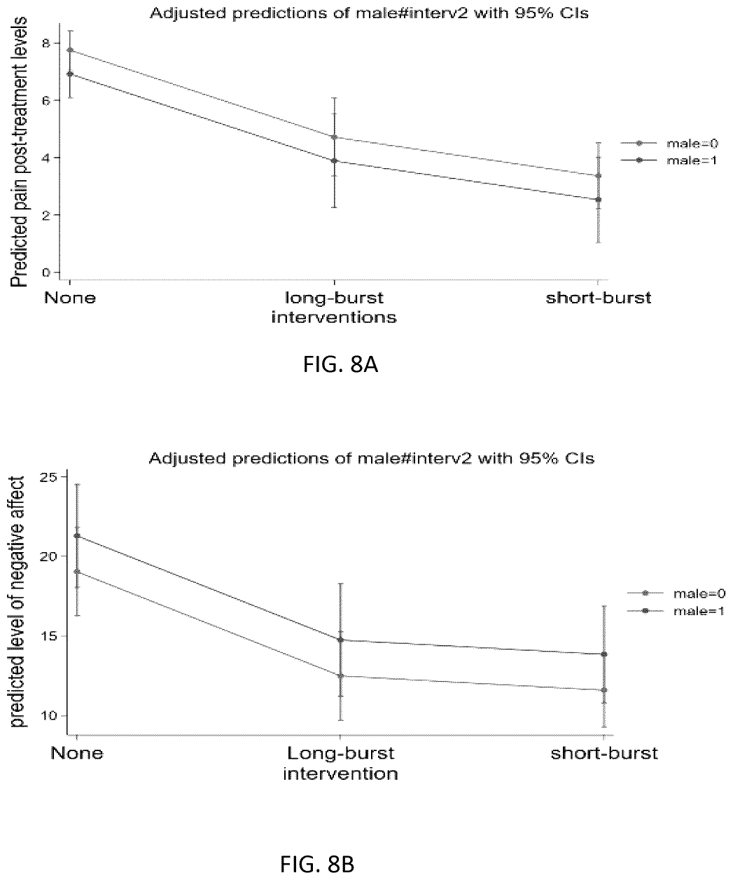

With respect to FIGS. 8A and 8B, graphs are provided that show that predicted pain and negative affective severity were more reduced in the short burst treatment groups compared to the long burst treatment groups, following linear mixed effect-coefficient analysis. FIG. 8A shows predicted levels of pain intensity regressed on the treatment group, adjusting for gender and other demographic covariates. As shown, treatment using the long burst mode and short burst mode reduced the predicted pain levels post treatment, with the short burst mode being more effective. FIG. 8B shows negative affect seven days post-treatment from linear mixed effect analysis of pre- and post-treatment measures regressed on the treatment group, adjusting for gender and other demographic covariates. As shown, treatment using the long burst mode and short burst mode reduced the predicted level of negative affect, with the short burst mode being more effective.

BCP is a suitable chemical for treating pain. BCP has molecular targets, CB1 and CB2, that belong to the endocannabinoid system of a mammalian body. BCP is a suitable chemical for treating pain because CB1 receptors are mainly distributed in the human brain, specifically in the cortex and hippocampus regions, which are responsible for cognitive, decision-making, mood regulation and CB2 agonists, which are primarily present in the peripheral nervous system, are critically involved in the modulation of inflammatory and pain responses. BCP is a Phyto-cannabinoid, obtained from plants, which selectively binds to peripheral cannabinoid receptors (CB2 receptors) and functions as a full agonist.

Testing was conducted to determine whether treatment with odorants including a 60% BCP content produces stronger and faster pain, affective, and cognitive responses in chronic pain patients. In this test, the short burst treatment method (as described above) was administered on 60 chronic pain patients where one group received short burst treatment where the odorants included 60% BCP while another group received short burst treatment without BCP (less than 1% BCP).

With reference to Table 5 below, the test results of Table 5 show the impact of the treatment with BCP and without BCP on post-treatment versus pre-treatment levels in patient reported pain intensity, patient reported pain interference, negative affect subscale of Positive and Negative Affect Schedule (PANAS), and sleep duration from the Pittsburgh Sleep Inventory (PSQI) from linear mixed effect models. As shown in Table 5, treatment without BCP significantly reduced severity of pain intensity, severity of pain interference with daily functions, and severity of negative affect. There is also a trend (P=0.051) towards increased sleep duration for treatment without BCP. These post-treatment to pre-treatment changes contrast with 14 days of observation of same individuals before treatment, when their pain, negative affect, and sleep measures were unchanged during the 14 days of pre-treatment observation.

The results shown in Table 5 further highlight the efficacy of treatment using odorants with BCP in improving severity of pain intensity, pain interference with daily functions, negative affect, and sleep impairments compared to treatment without BCP. As shown in the results, the coefficients of the treatment with BCP are larger than treatment without BCP, which demonstrates that BCP improves the effect of the treatment.

TABLE 5
Effect of Intervention on Pain, Affect and Sleep after 14 Days

| | Interven- | Coeffi- | Robust | | P |
|---|---|---|---|---|---|
| Outcome | tion | cient | SE | Z | value |
| Pain intensity | CBOT | −1.87 | 0.43 | −4.32 | <0.0001 |
| | CBOT-BCP | −4.09 | 0.32 | −12.86 | <0.00001 |
| Negative affect | CBOT | −5.06 | 1.47 | −3.43 | 0.001 |
| | CBOT-BCP | −8.70 | 1.23 | −7.08 | <0.0001 |
| Pain interference | CBOT | −3.84 | 0.89 | −4.32 | <0.0001 |
| | CBOT-BCP | −6.84 | 0.98 | −7.00 | <0.0001 |
| Sleep duration | CBOT | 0.68 | 0.35 | 1.95 | 0.051 |
| | CBOT-BCP | 0.73 | 0.31 | 2.37 | 0.018 |

Table 6 is a contingency table of the success of treatment with BCP, demonstrating that treatment with BCP for seven days reduces pain intensity by more than 30% (comparing day 14 to baseline scores) in 88% of patients treated using treatment with BCP. In contrast, 20 of these participants were followed for 14 days pre-treatment as control and none experienced ≥30% pain reduction.

TABLE 6
Proportion (%) of Population with 30% Pain Reduction

| 30% reduction in | CBOT-BCP | | |
|---|---|---|---|
| pain intensity | No | Yes | Total |
| No | 14 (56%) | 3 (12%) | 17 (34%) |
| Yes | 11 (44%) | 22 (88%) | 33 (66%) |
| Total | 25 (100%) | 25 (100%) | 50 (100%) |

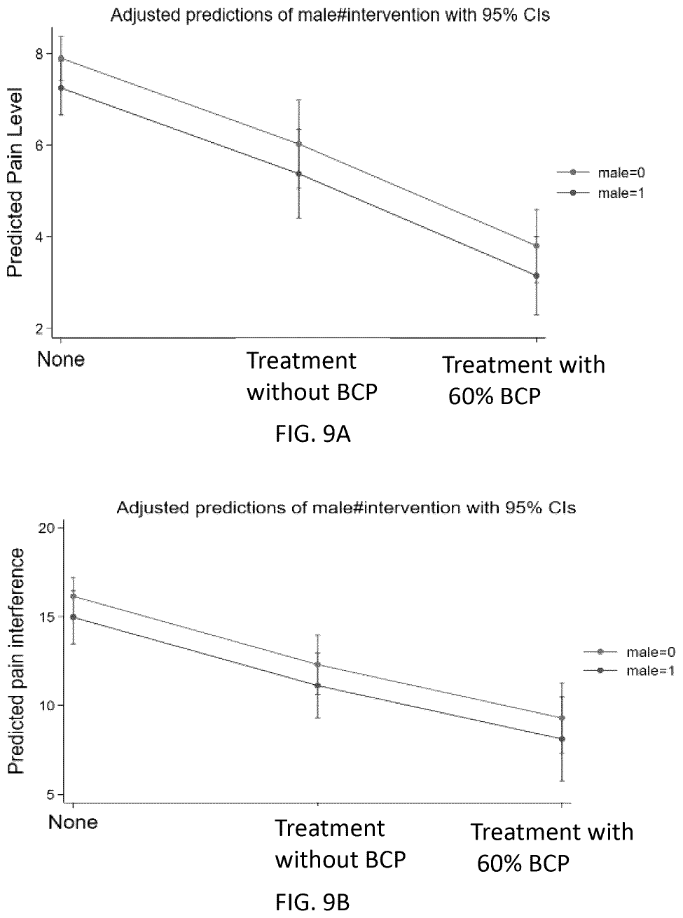

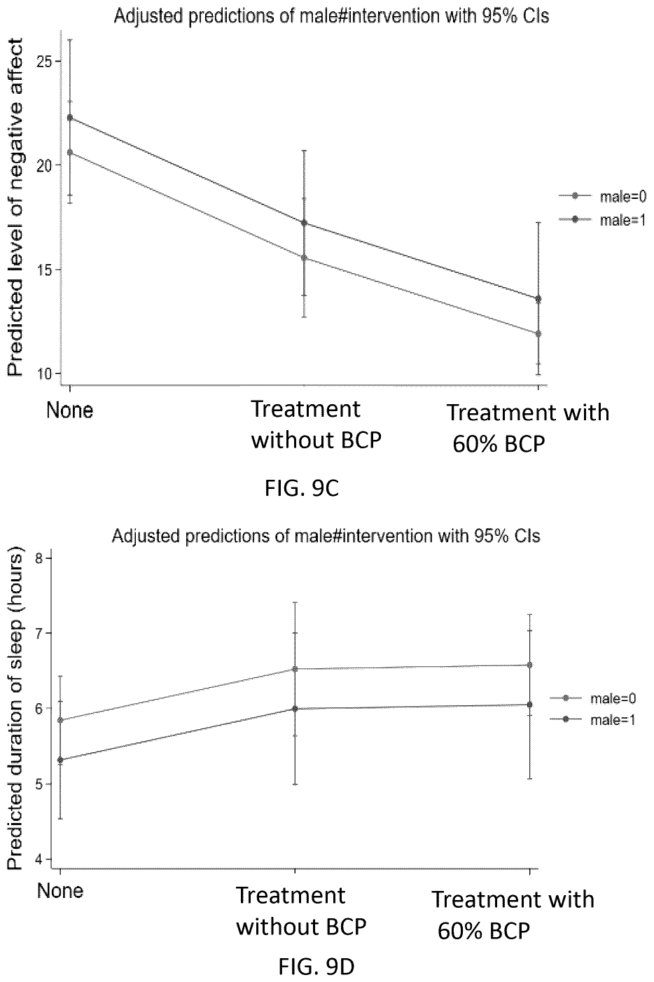

FIGS. 9A-9D respectively illustrate predicted levels of pain intensity, pain interference, negative affect, and sleep duration seven days after treatment with BCP from linear mixed effect analysis of pre-treatment and post-treatment measures regressed on treatment group, adjusting for gender and other demographic covariates. The results shown in FIGS. 9A-9D confirm the effectiveness of treatment with BCP.

The conclusion of this testing is that treatment without BCP and treatment with BCP using the short burst method both significantly reduce severity of pain and negative affect, but treatment with BCP is more effective in improving pain, negative affect and sleep. Also, the test results suggest that BCP adds sleep-enhancing effect to the pain-reducing effect of short burst stimulations.

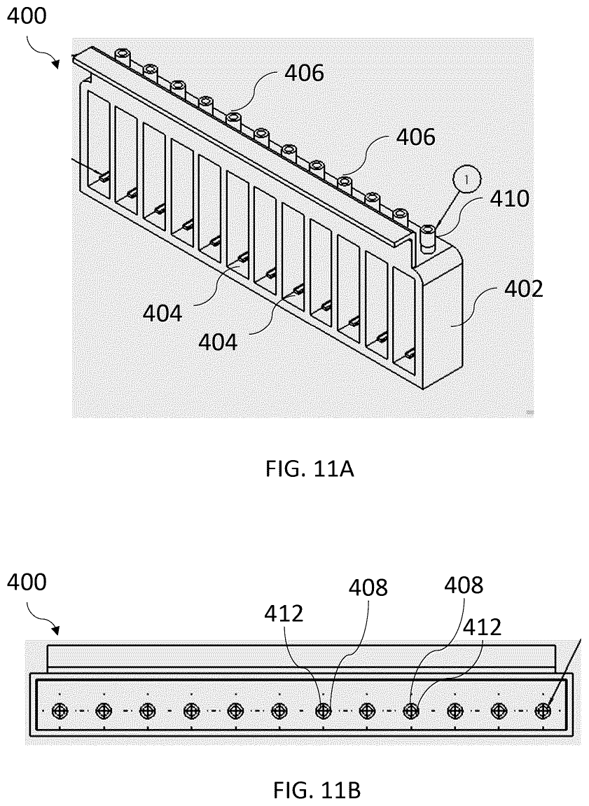

With respect to FIGS. 11A and 11B, an odor cartridge 400 is provided according to another embodiment that may be used with odor emitter similar to odor emitter 102. The odor cartridge 400 includes a carrier 402 including a plurality of odor chambers 404. The odor chambers 404 may each receive an odorant in the odor chamber 404. For example, each odor chamber may contain an absorbent material (e.g., cotton such as dental cotton rolls, gauze pads, microfiber fabric, sponges, or other materials having a porous structure to absorb and retain essential oils, and an odorant, such as an essential oil. Each odor chamber 404 may include an inlet 406 and an outlet 408. In the form shown, the inlet 406 is on one side of the carrier 402 and the outlet 408 is on the opposite side of the carrier 402. The pump 112 may force air through the chamber 404 from the inlet 406 to the outlet 408 to odorize the air. The inlets 406 may be connected to the odor emitter 102 to form a fluid tight connection with the odor emitter 102. For example, each inlet 406 of the odor cartridge 400 includes a tube connector 410 to which a flexible tube of the odor emitter 102 may be removably connected to form a fluid tight connection therebetween. In other forms, a seal (e.g., a gasket) of the odor cartridge 400 and/or odor emitter 102 is positioned about the inlets 406 and the inlets 406 pressed against the odor emitter with the seal therebetween to form a fluid tight connection.

The outlets 408 each include a one-way valve 412 to permit air flow out of the chamber 404 while inhibiting air flow into the chamber 404 through the outlet 408. The outlets 408 may be connected to the odor emitter 102 to form a fluid tight connection with the odor emitter 102. For example, each outlet 408 of the odor cartridge 400 may include a tube connector to which a flexible tube of the odor emitter 102 may be removably connected to form a fluid tight connection therebetween. In other forms, a seal (e.g., a gasket, o-ring) of the odor cartridge 400 and/or odor emitter 102 is positioned about the outlets 408 and the outlets 408 pressed against the odor emitter 102 with the seal therebetween to form a fluid tight connection.

In some forms, the odor cartridge includes an outlet manifold (e.g., integrally formed with the carrier 402) that the outlets 408 of the odor cartridge 400 connect to. The outlet manifold of the cartridge 400 may be connected to the odor emitter 102 such that air flows through the outlets 408 to the outlet manifold and from the outlet manifold to the odor emitter 102 where the odorant is directed to the cannula 104.

One or more walls may be removably connected to the carrier 402 to close the odor chambers 404 and form a fluid tight seal with the carrier 402 to inhibit fluid from leaking from the odor chambers 404. In one form, a single plate is attached to the carrier 402 to close all of the odor chambers 404 simultaneously. In one form, each odor chamber 404 has its own plate to close each odor chamber individually 404 and permit access to each odor chamber 404 individually. The plate(s) may be detached from the carrier 402 to clean and refill the cartridge. For example, once the cartridge 400 has been used, the odor chambers 404 may be accessed (e.g., the plate(s) removed) to clean the odor chambers 404 and refill them with odorants. The odor cartridge 400 may be reused once refilled with odorant.

In some forms, the odor cartridge 400 is formed such that the odor cartridge 400 is not refillable. The odor cartridge 400 may thus be a disposable cartridge that is discarded upon use. For example, the odor chambers 404 are permanently closed such that they cannot be accessed for refilling. For instance, the plate may be welded to the carrier 402 to inhibit removal of the plate.

In some forms, the odor cartridge 400 includes a memory chip that stores information about the odor cartridge 400 and/or the odorants in the odor cartridge 400. The information stored on the memory chip may be readable by the odor emitter 102. For instance, the odor cartridge 400 may include a radio-frequency identification (RFID) tag having the memory chip. The odor emitter 102 may include an RFID reader that reads the RFID tag of the cartridge when the cartridge is connected to the odor emitter 102. As examples, the memory chip of the cartridge may store information identifying the manufacturer of the cartridge, the type of odorants in the odor cartridge (e.g., which scents), and the locations of each odorant in the odor cartridge (e.g., which type of odorant is in each odor chamber). The odor emitter 102 may read or receive the information stored on the memory chip of the cartridge. For example, the odor emitter 102 may identify which scents are available to emit to the user during treatment. As another example, the odor emitter 102 may identify the manufacturer of the cartridge and inhibit operation of the odor emitter 102 when a cartridge from an unauthorized manufacturer is inserted (e.g., the odor emitter 102 may display an error to the user).

In some forms, the odor cartridge 400 is connected to a separate outlet manifold that includes the one-way valves 412 associated with each odor chamber 404. The outlet manifold may receive air flowing from each of the odor chambers 404 to collect the air into a single air passage.

In some forms, each odor chamber 404 of the odor cartridge 400 is separate from the other odor chambers. The carrier 402 of the odor cartridge 400 may be a frame that holds the odor chambers 404 together as a single unit.

Uses of singular terms such as “a,” “an,” are intended to cover both the singular and the plural, unless otherwise indicated herein or clearly contradicted by context. The terms “comprising,” “having” “including,” and “containing” are to be construed as open-ended terms. It is intended that the phrase “at least one of” as used herein be interpreted in the disjunctive sense. For example, the phrase “at least one of A and B” is intended to encompass A, B, or both A and B.

While there have been illustrated and described particular embodiments of the present invention, those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

What is claimed is:

1. An apparatus for treating pain in a patient, the apparatus comprising:
a plurality of odorant inputs to be connected to a plurality of odorant sources each having an outlet to permit odorant to flow from the associated odorant source;
a gas source operable to cause odorant to flow from the plurality of odorant inputs to the patient;
a user interface; and
a controller operably coupled to the plurality of odorant inputs, the gas source, and the user interface, the controller configured to:
operate the gas source and the plurality of odorant inputs to emit odorant from each of the plurality of odorant sources to a patient independently of the other odorant sources of the plurality of odorant sources; and
for each odorant source of the plurality of odorant sources, upon emitting odorant, prompt the patient to perform a cognitive task via the user interface.

2. The apparatus of claim 1 wherein each odorant input of the plurality of odorant inputs includes a control valve operable to permit odorant to flow from the associated odorant source; and
the controller is further configured to operate the control valve of each odorant input of the plurality of odorant inputs to cause odorant to be emitted from each of the plurality of odorant sources to the patient independently of the other odorant sources.

3. The apparatus of claim 2 wherein the control valve includes a solenoid operable by the controller to move the control valve between an open configuration and a closed configuration.

4. The apparatus of claim 1 further comprising a cartridge receiver to releasably receive a cartridge including the plurality of odorant sources.

5. The apparatus of claim 4 in combination with the cartridge, wherein each of the plurality of odorant sources include a tank containing odorant.

6. The apparatus of claim 1 wherein the controller is further configured to cease emission of odorant for a time period when switching between odorant sources of the plurality of odorant sources.

7. The apparatus of claim 6 wherein to cease emission of odorant includes closing each of the plurality of odorant inputs.

8. The apparatus of claim 6 wherein to cease odorant emission includes ceasing odorant emission for a time period in a range of about 10 seconds to 120 seconds.

9. The apparatus of claim 1 further comprising an gas outlet connectable to a cannula of the patient, the gas source operable to cause gas to flow toward the gas outlet.

10. The apparatus of claim 1 wherein the gas source includes at least one of a pump, an air blower, a compressor, and a pressurized gas tank.

11. The apparatus of claim 1 wherein the user interface includes a display screen, wherein to prompt the patient to perform the cognitive task includes prompting the patient via the display screen.

12. The apparatus of claim 1 wherein the user interface includes at least one of a touchscreen display and/or a button, wherein the controller is further configured to receive input from a patient in response to prompting the patient to perform the cognitive task.

13. The apparatus of claim 1 wherein to operate the gas source and the plurality of odorant inputs to emit odorant from each of the plurality of odorant sources independently of the other odorant sources includes opening an odorant input of the plurality of odorant inputs while closing the other odorant inputs of the plurality of odorant inputs.

14. The apparatus of claim 1 wherein to emit odorant from each of the plurality of odorant sources includes emitting odorant from each of the plurality of odorant sources for a time period in a range of about 5 seconds to 70 seconds.

15. The apparatus of claim 1 wherein the cognitive task includes identifying the odorant being emitted.

16. The apparatus of claim 1 wherein at least one of the plurality of odorant sources includes beta-caryophyllene.

17. The apparatus of claim 1 wherein to operate the gas source and the plurality of odorant inputs to emit odorant from each of the plurality of odorant sources includes emitting odorant from each odorant source of the plurality of odorant sources in a random order.

18. The apparatus of claim 1 wherein the plurality of odorant sources includes about 8 to 12 unique odorants having unique odorant profiles.

19. The apparatus of claim 1 wherein the controller is configured to operate in another phase where the controller operates the gas source and an odorant input of each of the plurality of odorant inputs to emit odorant from each of the plurality of odorant sources to the patient independently of other odorant sources of the plurality of odorant sources without prompting the patient to perform the cognitive task.

20. The apparatus of claim 1 further comprising a portable housing supporting the plurality of odorant inputs, the gas source, the user interface, and the controller.

21. The apparatus of claim 1 further comprising a sensor to measure a physiological parameter of the patient.

22. The apparatus of claim 1 further comprising communication circuitry operably coupled the controller, the controller configured to communicate data including treatment session data to a remote computer accessible by a clinician.

23. A method of treating pain in a patient, the method comprising:
in one phase:
emitting each odorant of a plurality of odorants to a patient independently of other odorants of the plurality of odorants; and
upon emitting each odorant of the plurality of odorants, prompting the patient to perform a cognitive task that activates an olfactory cortex of a brain of the patient to effect neuroplastic changes to the olfactory cortex to reduce pain in the patient.

24. The method of claim 23 wherein emitting each odorant includes:
nebulizing an odorant; and
operating an airflow generator to cause the odorant to flow to the patient.

25. The method of claim 23 wherein emitting each odorant includes opening a valve associated with an odorant to be emitted.

26. The method of claim 23 wherein emitting each odorant to the patient activates a olfactory cortex of a brain of the patient to achieve at least a 3% peak activation.

27. The method of claim 26 wherein the activation of the olfactory cortex from the odorant and the cognitive task effects neuroplastic changes to the olfactory cortex.

28. The method of claim 23 further comprising detecting a physiological parameter of the patient.

29. The method of claim 23 wherein the plurality of odorants each include an essential oil having a unique odorant profile from the essential oils of the others of the plurality of odorants.

30. The method of claim 29 wherein the plurality of essential oils includes one or more of:
banana oil;
vanilla oil;
lemon oil;
cinnamon oil;
rosemary oil;
eucalyptus oil;
ginger oil;
mint oil;
orange oil;
citrus oil;
clove oil; and
lavender oil.

31. The method of claim 23 wherein each odorant of the plurality of odorants is emitted for a time period in a range of about 5 seconds to about 70 seconds.

32. The method of claim 23 wherein each odorant of the plurality of odorants is emitted for about 10 seconds.

33. The method of claim 23 further comprising ceasing odorant emission between emission of each odorant of the plurality of odorants.

34. The method of claim 33 wherein ceasing odorant emission includes ceasing odorant emission for a time period in a range of about 25 seconds to 65 seconds.

35. The method of claim 23 wherein the cognitive task includes identifying which odorant of the plurality of odorants is being emitted.

36. The method of claim 23 further comprising receiving input from the patient in response to prompting the patient to perform the cognitive task.

37. The method of claim 23 wherein at least one of the plurality of odorants includes beta-caryophyllene.

38. The method of claim 37 wherein each odorant of the plurality of odorants has a composition of beta-caryophyllene in a range of about 10% to about 80% by volume.

39. The method of claim 38 wherein each odorant of the plurality of odorants has a composition of beta-caryophyllene in a range of about 30% to about 60% by volume.

40. The method of claim 23 wherein emitting each odorant of the plurality includes emitting each odorant of the plurality in a random order.

41. The method of claim 23 wherein the plurality of odorants includes about 8 to 12 unique odorants having unique odorant profiles.

42. The method of claim 23 wherein in the one phase each of the plurality of odorants are emitted a single time.

43. The method of claim 23 further comprising another phase including:
emitting each odorant of the plurality of odorants to the patient independently of other odorants of the plurality without prompting the patient to perform the cognitive task.

44. The method of claim 23 wherein the method includes four phases and the plurality of odorants includes ten odorants, wherein in each phase each odorant of the plurality of odorants is emitted a single time.
